(19) United States
(12) Patent Application Publication
(10) Pub. No.: US 2025/0387248 A1
(43) Pub. Date: Dec. 25, 2025

(54) STENT EXPANDING BALLOON WITH FRICTION ENHANCING MATERIALS AND FEATURES

(71) Applicant: Merit Medical Systems, Inc., South Jordan, UT (US)

(72) Inventors: Wayne Mower, Bountiful, UT (US); Adam Wilhite, Herriman, UT (US); Jason Wiersdorf, West Jordan, UT (US); Joanne Hislop, American Fork, UT (US); Mattias Nielsen, North Salt Lake, UT (US); John Hall, Bountiful, UT (US)

(21) Appl. No.: 19/244,226

(22) Filed: Jun. 20, 2025

Related U.S. Application Data
(60) Provisional application No. 63/662,284, filed on Jun. 20, 2024.

(51) Int. Cl. A61F 2/958; A61M 25/10; B29C 69/02; B29L 31/00

(52) U.S. Cl. CPC A61F 2/958; A61M 25/1029; A61F 2002/9583; A61M 2025/1031; A61M 2205/0238; B29C 69/02; B29L 2031/7534

(57) ABSTRACT

Medical balloons and methods used to retain and expand a stent within a stenosis or stricture of a bodily passage are disclosed. The balloons include a base material layer and a friction enhancing feature coupled to an exterior surface of the base material layer. The friction enhancing feature includes a second material layer disposed over the exterior surface to form a laminar formed balloon. The second material layer is more compressible than the base material layer to provide an increased static stent holding force.

### RELATED CASES

This application claims priority to U.S. Provisional Application No. 63/662,284, filed on Jun. 20, 2024, and titled “STENT EXPANDING BALLOON WITH FRICTION ENHANCING MATERIALS AND FEATURES,” which is hereby incorporated by reference in its entirety.

### TECHNICAL FIELD

The present disclosure relates generally to medical devices to treat a stenosis or stricture of a bodily passage. More specifically, the present disclosure relates to a balloon to expand a stent placed within the stenosis or stricture of the bodily passage. More specifically, the present disclosure relates to a balloon with friction enhancing features to increase the coefficient of friction of the balloon.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a tube of an embodiment of a stent expanding balloon according to one embodiment of the present disclosure.

FIG. 2 is a perspective view of an embodiment of a laminar structure of the tube of FIG. 1.

FIG. 3 is a perspective view of an embodiment of a formed laminar balloon of the laminar structure of FIG. 2.

FIG. 4 is a side cross-sectional view of the formed laminar balloon of FIG. 3.

FIG. 5 is a side view of an embodiment of a stent expanding balloon having a friction enhancing feature dispersed over an external surface according to one embodiment of the present disclosure.

FIG. 6A is a side view of a detailed portion of the stent expanding balloon of FIG. 5 showing an embodiment of the friction enhancing feature.

FIG. 6B is a side view of a detailed portion of the stent expanding balloon of FIG. 5 showing another embodiment of the friction enhancing feature.

FIG. 7 is a side view of an embodiment of a stent expanding balloon having a first coating and a second coating according to one embodiment of the present disclosure.

### DETAILED DESCRIPTION

In certain instances, a stenosis or stricture may form in a bodily passage, such as a blood vessel, a gastrointestinal tract, etc. The stenosis or stricture may restrict fluid flow (blood, digestive fluids, etc.) within the bodily passage resulting in morbid or mortal complications. The stenosis or stricture can be treated by performing an angioplasty in which an inflatable balloon is positioned within the stenosis or stricture and the balloon in inflated to help open the stenosis or stricture and open blood flow. In some situations, during the angioplasty a stent may be placed within the stenosis or stricture using a stent delivery device having a stent expanding balloon. When the stent is positioned, the stent expanding balloon can be inflated causing the stent to expand to open the stenosis or stricture. In some embodiments, the stent expanding balloon has a slick exterior surface and the stent may not be adequately coupled or secured to the stent expanding balloon during positioning of the stent resulting “watermelon seeding” or balloon slippage. Balloon slippage can result in premature displacement of the stent from the stent expanding balloon. This may result in improper positioning or expansion of the stent. In other embodiments, the balloon expanding stent inflates from both ends prior to inflating a middle section forming a dog bone shaped inflated balloon. This may cause the stent to accordion from one or both ends toward a middle portion resulting in a shortened stent.

Embodiments herein describe balloons, such as stent expanding balloons, with friction enhancing features and methods of manufacturing balloons with friction enhancing features to increase static stent holding forces of the balloon. In some embodiments within the scope of this disclosure, the balloons include a first tapered end portion; a second tapered end portion; and a body portion disposed between the first tapered end portion and the second tapered end portion. The body portion can include an exterior surface and an interior surface, wherein the exterior surface may include a friction enhancing feature configured to increase a static stent holding force. In certain embodiments, the balloons may have a static stent holding force that is greater than five Newtons. A coefficient of friction of the exterior surface is greater than a coefficient of friction of the interior surface. In another embodiment, the body includes a first material layer defining the interior surface and the friction enhancing feature includes a second material layer disposed over the first material layer. The second material layer is more compressible than the first material layer. A material of the second material layer has a Shore durometer of between 50 A and 80 D. A thickness of the second material layer ranges between 0.008 millimeter and 0.051 millimeter. The second material layer is one or more of silicone, polyurethane, polyether block amide, polytetrafluoroethylene, nylon, acrylic, and methacrylic. In another embodiment, the second material layer is a sleeve disposed over the first material layer. Fiction enhancing features may include textures, geometry, or any topological feature of a surface of a balloon. In some such embodiments, the friction enhancing features include a plurality of micro or nano structures extending radially outward from the exterior surface. In one embodiment, the structures include a seta having a projection. In another embodiment, the structures include a nano tube. Each of the structures provide an electrostatic force comprising an attractive van der Waals force of at least 0.4 μN.

In certain embodiments, a method of forming the balloons include the steps of: extruding a first material to form a tube comprising a first material layer having an interior surface and an exterior surface; disposing a second material layer over the exterior surface of the medical balloon, wherein the second material layer is coupled to the exterior surface; and blow molding the tube in a mold to form a medical balloon. In some embodiments, the exterior surface is treated using one or more of dielectric barrier discharge, chemical priming, and mechanical texturing, prior to the step of disposing the second material layer over the exterior surface of the tube. After treatment, the exterior surface has a water contact angle of less than 50 degrees. In another embodiment, the second material layer is disposed over the exterior surface of the tube by co-extruding the second material layer over the first material layer of the tube. The second material layer includes one or more of polyurethane, silicone, polyether block amide, acrylic, and methacrylic. In another embodiment, the second material layer is disposed over the exterior surface of the tube by dipping the tube into a solvated polymer solution. The solvated polymer solution includes one or more of polyurethane, silicone, polyether block amide, acrylic, and methacrylic.

The friction enhancing features of the balloons described herein may be used to help prevent “watermelon seeding” of a stent and/or any type balloon slippage during a procedure, that is, balloon slippage with respect to a stent or balloon slippage with respect to anatomical features during procedures without a stent. The balloons described herein may be used in a variety of different situations. As discussed above, the balloons described herein may be used in angioplasty procedure to treat stenosis or strictures, with or without a stent. The balloons described herein may also be used in the placement of balloon expandable values, such as mitral valve replacements. The balloons described herein may be used to place stents throughout a patient's body, such as the vasculature, spine, esophagus, and the like. Still further, the balloons described herein may be used to treat blood vessels or other structures without the use of a stent, including compressing plaque, stretching valves, delivering one or more medicaments, and so forth.

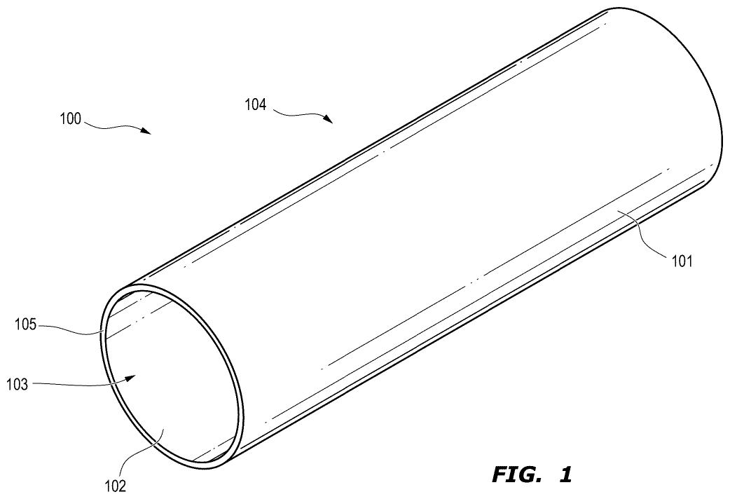

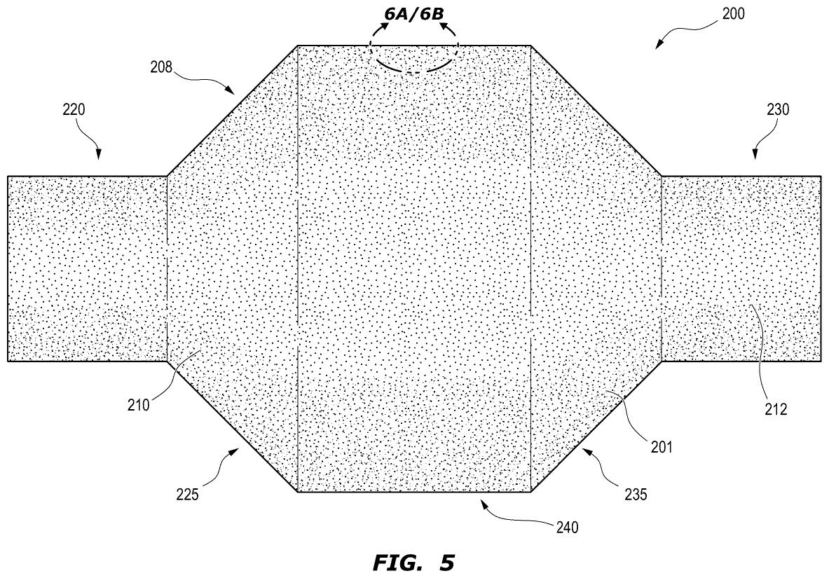

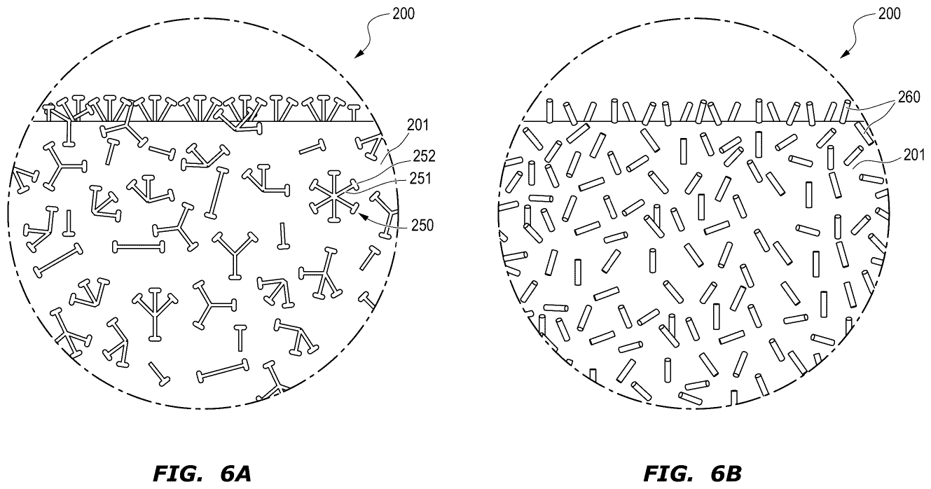

FIGS. 1-4 illustrate an embodiment of an embodiment of a stent expanding balloon having one or more friction enhancing features including, in some embodiments, a laminar structure or use of layers or other features. FIG. 5 illustrates an embodiment of a stent expanding balloon having a friction enhancing feature dispersed over an external surface. FIG. 6A illustrates a detailed portion of the stent expanding balloon of FIG. 5 showing an embodiment of the friction enhancing feature having a plurality of micro or nano sized setae and projections or protrusions. FIG. 6B illustrates a detailed portion of the stent expanding balloon of FIG. 5 showing another embodiment of the friction enhancing feature having a plurality of micro or nano sized tubes or columns. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

As illustrated in FIG. 1, a balloon, which may be configured as a stent expanding balloon 100 includes an extruded tube 104 having a first material layer or base wall 105. The first material layer 105 has an exterior surface 101 and an interior surface 102 defining a bore 103 extending through the length of the tube 104. In the depicted embodiment, the tube 104 has a circular cross-sectional shape. Other cross-sectional shapes are within the scope of this disclosure. The first material layer 105 may be formed from any suitable thermoplastic material to provide a compliant, semi-compliant, or non-compliant stent expanding balloon 100. For example, materials such as nylon, polyether block amide, and polyethylene terephthalate may be used. Other materials are within the scope of this disclosure.

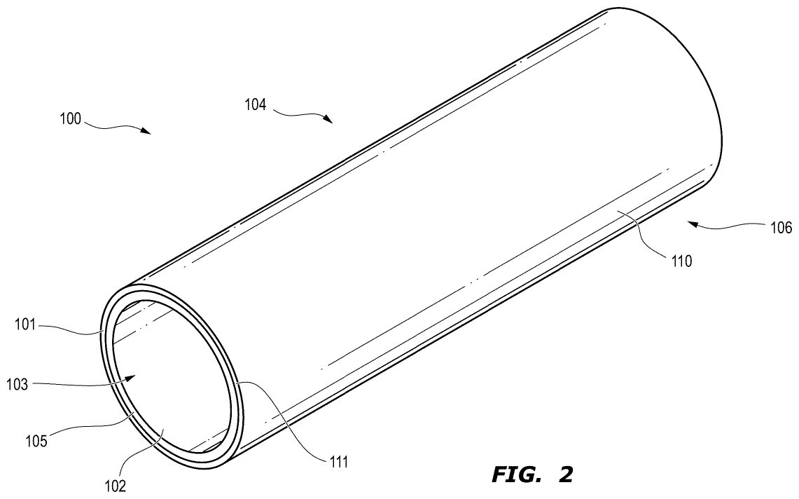

FIG. 2 illustrates the tube 104 of the stent expanding balloon 100 having a friction enhancing feature 110 disposed over and coupled to the exterior surface 101. As depicted, the friction enhancing feature 110 is a second material layer 111 disposed over and coupled to the exterior surface 101 to form a laminar structure 106. The second material layer 111 may be formed from any suitable material, such as silicone, polyurethane, polyether block amide, polytetrafluoroethylene, nylon, acrylic, and methacrylic. Other materials are within the scope of this disclosure. The second material layer 111 may have a thickness ranging between about 0.0008 millimeter and about 0.051 millimeter. The second material layer 111 may have a first coefficient of friction that is higher or greater than a second coefficient of friction of the first material layer 105. The first coefficient of friction may provide a static stent holding force of at least 5 N to ensure stability of a stent over the stent expanding balloon 100 when the stent is being positioned for deployment within a stenosis or stricture of a bodily passage, such as a blood vessel, a gastrointestinal tract, etc.

In certain embodiments, the second material layer 111 can be more compressible than the first material layer 105. The second material layer 111 may be formed from any suitable compressible material, such as silicone, polyurethane, and polyether block amide. Other materials are within the scope of this disclosure. The second material layer 111 may have a Shore hardness durometer of from about 50 A to about 80 D, and may be about 70 A, including from about 70A to 100 A.

In some embodiments, the second material layer 111 is disposed over the first material layer 105 and coupled to the exterior surface 101 to form the laminar structure 106 using a co-extrusion manufacturing technique. For example, pellets of the first material are melted in a first extruder. Pellets of the second material are melted in a second extruder. The melted first and second materials flow together with partial mixing at an interface between the materials. The combined materials are extruded through a die under pressure to form the first material layer 105 and the second material layer 111 of the laminar structure 106. In certain embodiments, the first and second materials may have similar melt indices to allow the first and second material layers to melt together to form a melt bond between the first material layer 105 and the second material layer 111.

In another embodiment, the second material layer 111 is disposed over the first material layer 105 and coupled to the exterior surface 101 to form the laminar structure 106 using a dip coating manufacturing technique. For example, the tube 104 is dipped into a solvated solution of the material of the second material layer 111 and a suitable solvent. When the tube 104 is removed from the solvated solution, the exterior surface 101 is coated with the solvated solution. The thickness of the coating may be determined by such factors as viscosity of the solvated solution and withdrawal rate of the tube 104 from the solvated solution. For example, a solvated solution having a high viscosity may form a thicker coating than a solvated solution having a low viscosity and a process having a fast withdrawal rate may form a thinner coat than a process having a slow withdrawal rate. The solvent evaporates from the coating leaving the second material to form the second material layer 111. In some embodiments, the solvent of the solvated solution may partially dissolve or soften the first material layer 105 to provide a solvent bond between the first material layer 105 and the second material layer 111.

In an embodiment, the exterior surface 101 of the tube 104 can be treated (i.e., cleaned or activated) prior to dip coating to enhance bonding between the first material layer 105 and the second material layer 111. The exterior surface 101 may be treated using any suitable technique to provide a water contact angle of less than 50 degrees. For example, the treatment may use dielectric barrier discharge (i.e., plasma or corona) or chemical priming.

In another embodiment, the second material layer 111 may include a sleeve disposed over the tube 104. The sleeve may include an internal surface coated with an adhesive.

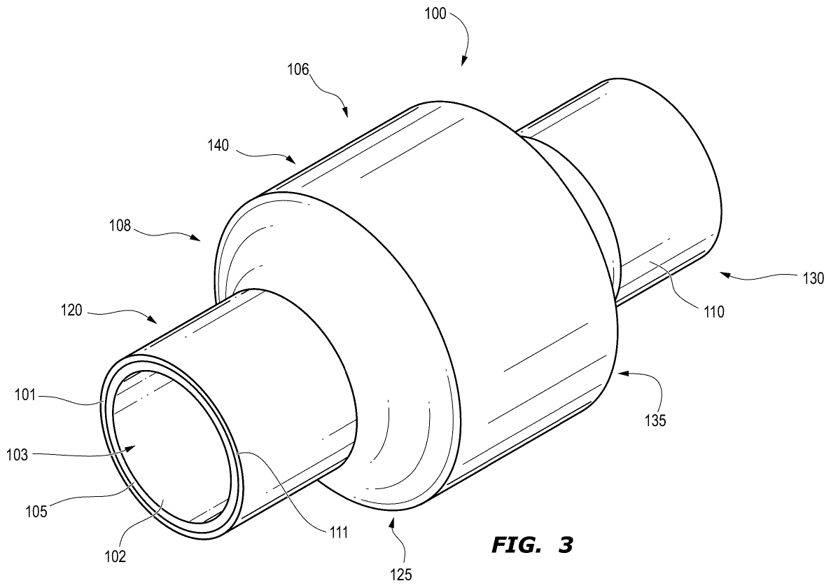

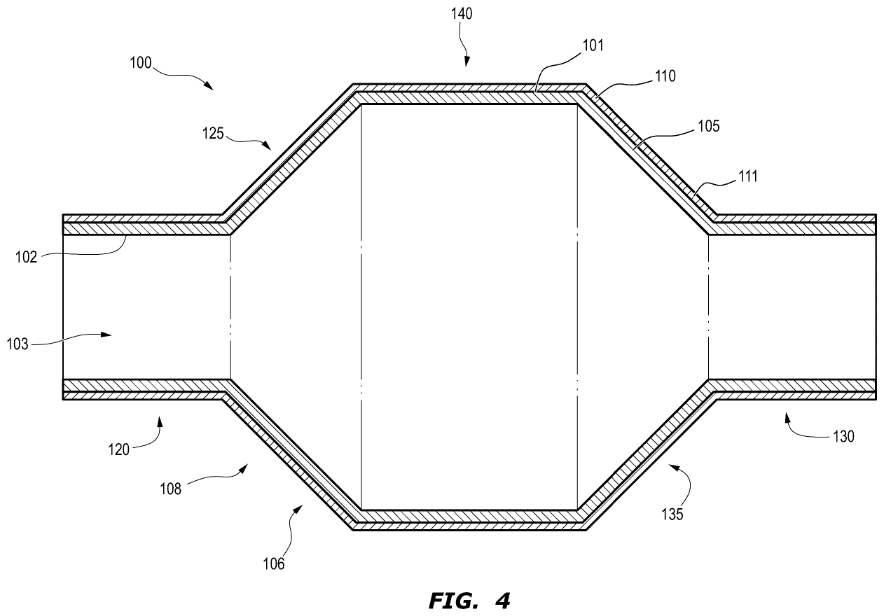

FIGS. 3 and 4 illustrate a formed laminar balloon 108 of the stent expanding balloon 100 formed from the laminar structure 106. As shown in FIG. 3, the formed laminar balloon 108 includes a first end portion 120, a second end portion 130, a middle portion 140 disposed between the first end portion 120 and the second end portion 130, and the friction enhancing feature 110. The bore 103 is defined by the interior surface 102 and can extend through the first end portion 120, the middle portion 140, and the second end portion 130 such that the first end portion 120, the middle portion 140 and the second end portion 130 are in fluid communication. An opening is disposed at each end of the bore 103. In some embodiments, the outer diameter of the first end portion 120 is substantially equivalent to the outer diameter of the second end portion 130. In other embodiments, the outer diameter of the first end portion 120 is different (i.e., smaller or larger) than the outer diameter of the second end portion 130.

A first taper region 125 may be disposed between the first end portion 120 and the middle portion 140. A second taper region 135 may be disposed between the second end portion 130 and the middle portion 140. In some embodiments, the length of the first taper region 125 is substantially equivalent to the length of the second taper region 135. In other embodiments, the length of the first taper region 125 is different (i.e., shorter or longer) than the length of the second taper region 135.

In certain embodiments, the formed laminar balloon 108 is formed by disposing the laminar structure 106 within a cavity of a heated mold having a desired stent expanding balloon shape, such as the balloon shape illustrated in FIG. 3. Alternative balloon shapes are within the scope of the present disclosure, such as cylindrical, conical, square, spherical, conical/square, conical/square extended, conical/spherical extended, extended spherical, tapered, dog bone, stepped, offset, conical/offset, and the like. Air pressure is applied to the bore 103 of the laminar structure 106. Heating of the materials of the first material layer 105 and the second material layer 111 causes the materials to soften. The air pressure causes the first material layer 105 and the second material layer 111 to expand radially outward to conform to a shape of the cavity and to form the formed laminar balloon 108. In some embodiments, a surface of the cavity may be coated with a release agent to prevent sticking of the second material layer 111 to the cavity surface. The first material layer 105 and the second material layer 111 are stretched resulting in a thinning of the layers 105, 111.

Throughout this disclosure, reference is made to laminar balloons or laminar structures wherein a balloon wall may be formed by layers of material. Embodiments wherein a single layer forms the balloon wall are likewise within the scope of this disclosure and, as used herein, any reference to a laminar structure or laminar balloon can include embodiments with a single layer of material or layers formed by any means (e.g. coextrusion, mechanical bonding, and so forth).

FIGS. 5-6B depict an embodiment of a balloon, which may be configured as a stent expanding balloon 200 that resembles the stent expanding balloon 100 described above in certain respects. Accordingly, like features are designated with like reference numerals, with the leading digit incremented to “2.” For example, the embodiment depicted in FIGS. 5-6B includes a friction enhancing feature 210 that may, in some respects, resemble the friction enhancing feature 110 of FIG. 2. Relevant disclosure set forth above regarding similarly identified features thus may not be repeated hereafter. Moreover, specific features of the stent expanding balloon 100 and related components shown in FIGS. 1-4 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the stent expanding balloon 200 and related components depicted in FIGS. 5-6B. Any suitable combination of the features, and variations of the same, described with respect to the stent expanding balloon 100 and related components illustrated in FIGS. 1-4 can be employed with the stent expanding balloon 200 and related components of FIGS. 5-6B, and vice versa. This pattern of disclosure applies equally to further embodiments depicted in subsequent figures and described hereafter, wherein the leading digits may be further incremented.

FIG. 5 illustrates another embodiment of a balloon, which may be configured as a stent expanding balloon 200. As depicted the stent expanding balloon 200 includes a formed balloon 208 defined by an outer surface 201. The formed balloon 208 includes a first portion 220, a second portion 230, a middle portion 240 disposed between the first portion 220 and a second portion 230, a first taper region 225 disposed between the first portion 220 and the middle portion 240, and a second taper region 235 disposed between the second portion 230 and the middle portion 240. The outer surface 201 includes a friction enhancing feature 210 dispersed over the outer surface 201. In another embodiment, the friction enhancing feature 210 is dispersed over the outer surface of the middle portion 240, the first taper region 225, and the second taper region 235. In some embodiments, the friction enhancing feature 210 is dispersed only over the outer surface 201 of the middle portion 240.

The friction enhancing feature 210 includes a plurality of micro or nano sized structures 212 extending from or into the outer surface 201. The structures 212 may be of any suitable shape or form that provides an adequate positive van der Waals force to cumulatively provide a static stent holding force of at least five Newtons when a stent is crimped around a deflated balloon. For example, FIG. 6A illustrates an embodiment of micro or nano sized structures 250 dispersed over the exterior surface 201. As depicted, the each of the structures 250 include a seta or hair 251 and a projection or protrusion 252 extending from an end of the seta 251. The projection 252 may be of any suitable shape, such as a spatula or mushroom. Other suitable shapes are within the scope of this disclosure. In some embodiments, the structures 250 include a clump or plurality of setae 251 and projections 252 clustered together. The projection 252 can provide a positive van der Waals force of about 0.4 μN. A plurality of structures 250 dispersed over the outer surface 201 can provide a stent static holding force of at least five Newtons when a stent is crimped around a deflated balloon.

FIG. 6B illustrates another embodiment of micro or nano sized structures 260 dispersed over the exterior surface 201. As depicted, the structures 260 include a tube or column extending from the exterior surface 201. Each structure 260 can provide a positive van der Waals force of about 0.4 μN. A plurality of structures 260 dispersed over the outer surface 201 can provide a stent static holding force of at least five Newtons when a stent is crimped around a deflated balloon.

In certain embodiments, the friction enhancing feature 210 is formed and dispersed over the outer surface 201 during the blow molding process of forming the formed balloon 208. A negative or cavity of the structures 212 can be provided in the cavity of the blow mold such that when the tube 104 is heated and pressurized within the blow mold the material of the first material layer 105 flows into the negative or cavity of the structures 212 to form the structures 212. In another embodiment, the structures 212 are dispersed over the outer surface 201 prior to the blow molding process.

In some embodiments, the friction enhancing feature 210 is formed and dispersed over the outer surface 201 by forming the structures 212 on a flexible film or tape and then wrapping the formed balloon 208 with the flexible film or tape.

In one embodiment, the structures 212 are formed by roughening the outer surface 201 to increase a surface area of the outer surface 201. The outer surface 201 may be roughened using any suitable technique such as sanding, sandblasting, peening, plasma etching, corona etching, electrochemical etching, or chemical etching. Other roughening techniques are within the scope of this disclosure.

For example, the outer surface 201 may be roughened to form friction enhancing features by sandblasting the outer surface 201 with a stream of abrasive material, such as aluminum oxide, crushed glass, glass beads, plastic, silicon carbide, pumice, steel shot, steel grit, organic compounds, and the like. In some embodiments, the size of each particle of the abrasive material may range from 20 microns to 100 microns. In some embodiments, the entire outer surface 201 may be roughened to form the friction enhancing features. In other embodiments, only select portions of the outer surface 201 is roughened to form friction enhancing features.

Furthermore, in some embodiments the outer surface 201 is roughened, sculpted, or otherwise imparted with surface features by a mold used to form the balloon 200. For example, in certain embodiments, the formed balloon 208 is formed by blow molding or other molding techniques. In some such embodiments, the material configured to form the balloon 208 is disposed within a cavity of a mold having a desired balloon shape, such as the balloon shape illustrated in FIG. 5. Alternative balloon shapes are within the scope of the present disclosure, such as cylindrical, conical, square, spherical, conical/square, conical/square extended, conical/spherical extended, extended spherical, tapered, dog bone, stepped, offset, conical/offset, and the like. Air pressure may then be applied to the material configured to form the formed balloon 208. In some embodiments, a tubular piece of material is disposed within a heated mold and pressure applied with the lumen of the tubular piece of material to expand the lumen into to mold cavity for form balloon 208. Heating of the material causes the material to soften. The air pressure causes the material of the stent expanding balloon to expand radially outward to conform to a shape of the cavity and to form the formed balloon 208. In some embodiments, a surface of the cavity may be coated with a release agent to prevent sticking of the formed balloon 208 to the cavity surface. The material of the formed balloon 208 may be stretched, resulting in a thinning of the material wall.

In some embodiments, the interior of the cavity of the mold may be roughened, formed, designed, or treated so that the outer surface 201 of the formed balloon 208 acquires a texture from the interior of the cavity of the mold. The interior of the mold may be roughened or otherwise modified using any suitable technique such as sanding, sandblasting, peening, plasma etching, corona etching, electrochemical etching, or chemical etching. Other roughening techniques are within the scope of this disclosure.

For example, the interior of the mold may be roughened by sandblasting the interior of the mold with a stream of abrasive material, such as aluminum oxide, crushed glass, glass beads, plastic, silicon carbide, pumice, steel shot, steel grit, organic compounds, and the like. In some embodiments, the size of each particle of the abrasive material may range from 20 microns to 100 microns. In some embodiments, the entire interior of the mold may be roughened so that outer surface 201 of the formed balloon 208 acquires friction enhancing features from the roughened interior of the mold during the molding process. In other embodiments, only select portions of the interior is roughened so that only portions of the outer surface 201 of the formed balloon 208 acquire friction enhancing features from the roughened interior of the mold during the molding process.

In some embodiments, features may be etched or carved into the interior of the mold. The features may have a design or pattern, similar to the micro or nano sized structures 250 and 260 illustrated in FIGS. 6A and 6B. The outer surface 201 of the formed balloon 208 may acquire the features etched or carved into the interior of the mold during the molding process. The present disclosure is not limited to the micro or nano sized structures 250 and 260 illustrated in FIGS. 6A and 6B, but may include additional micro or nano sized structures. In some embodiments, the entire interior of the mold may be etched or carved with features so that outer surface 201 of the formed balloon 208 acquires friction enhancing features from the interior of the mold of the etched or carved features during the molding process. In other embodiments, only select portions of the interior may be etched or carved with features so that outer surface 201 of the formed balloon 208 acquires friction enhancing features from the roughened interior of the mold of the etched or carved features during the molding process.

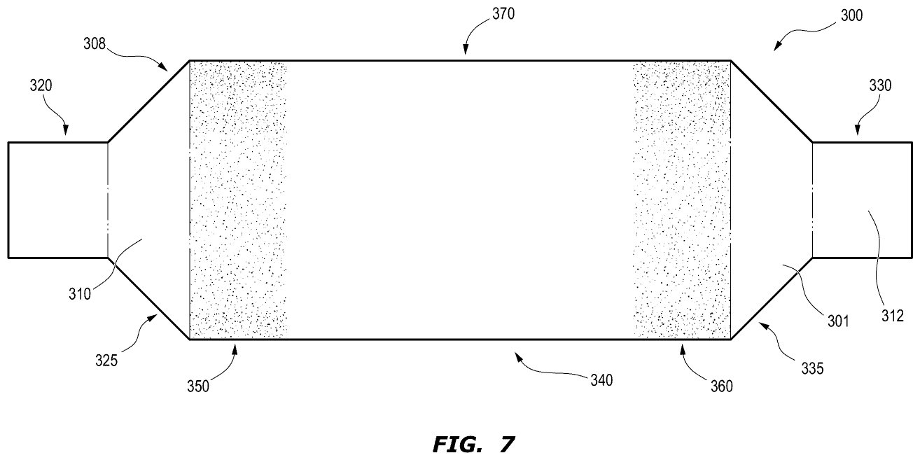

FIG. 7 illustrates another embodiment of a balloon, which may be configured as a stent expanding balloon 300. As depicted, the stent expanding balloon 300 includes a formed balloon 308 defined by an outer surface 301. The formed balloon includes a first portion 320, a second portion 330, a middle portion 340 disposed between the first portion 320 and a second portion 330, a first taper region 325 disposed between the first portion 320 and the middle portion 340, and a second taper region 335 disposed between the second portion 330 and the middle portion 340.

The outer surface 301 comprises friction enhancing features which include a pair of coating portions, coatings 350 and 360, disposed at opposing ends of the middle portion 340. In the illustrated embodiments, a first coating 350 is disposed at a first end of the middle portion 340 near the first taper region 325 and a second coating 360 is disposed a second end of the middle portion 340 near the second taper region 335. The middle portion 340 may comprise an uncoated portion 370 disposed between the first coating 350 and the second coating 360. In some embodiments, a length of the first coating 350 may be the same as a length of the second coating 360. In some embodiments, a length of the first coating 350 may be longer than a length of the second coating 360. In some embodiments, a length of the first coating 350 may be shorter than a length of the second embodiment.

The first coating 350 and the second coating 360 have a durometer that is different from the durometer of the uncoated portion 370. The durometer of the first coating 350 and the second coating 360 may have durometers within any of the ranges of durometers disclosed herein for friction enhancing features, including from about 50 A to about 80 D, may be about 70 A, may be from about 70A to 100 A, and may be around 50 D. The difference in durometer between the first coating 350 and the second coating 360 compared to durometer of the uncoated portion 370 may increase the coefficient of friction of the first coating 350 and the second coating compared to the coefficient of friction of the uncoated portion 370.

In some embodiments, the first coating 350 is configured to engage with a first end of a stent and the second coating 360 is configured to engage a second end of the stent to reduce displacement (accordioning, slipping, watermelon seeding, and so forth) of the stent during the expansion of the stent. The coatings 350, 360 may increase the coefficient of friction between the stent and the balloon along the coated portions. The increased coefficient of friction enables the first coating 350 to better engage with the first end of the stent and the second coating 360 to better engage with the second end of the stent and prevent displacement of the stent with respect to the balloon during deployment of the stent.

In some embodiments, including embodiments wherein the balloon is utilized for treatment of a bodily structure without a stent, the first coating 350 may be configured to engage with a first portion of a stenosis or stricture and the second coating 360 may be configured to engage with a second portion of a stenosis of stricture. The increased coefficient of friction enables the first coating 350 to better engage with the first end of the stenosis or stricture and the second coating 360 to better engage with the second end of the stenosis or stricture and prevent displacement of the balloon with respect to the anatomy to be treated during treatment of the stenosis or stricture.

In some embodiments, the first coating 350 and the second coating 360 may be polymeric, elastomeric, and/or pliable. The coatings 350, 360 may be sprayed on, glued on, heat bonded, mechanically bonded, and so forth. In some embodiments, the coatings 350, 360 may comprise a substance such as a flowable polymer, including curable flowable polymers such as glues, that is applied to the balloon and cured. In some instances, polymeric glues may be an acrylic or methacrylic glue. The polymeric glues may be manufactured by Loctite®, including Loctite 3953, may be used for the coatings 350, 360.

In some embodiments, the coating may be disposed in a variety of different locations on the stent expanding balloon 300. For example, in some embodiments a third coating may be disposed in the center of the middle portion 340 in between the first coating 350 and the second coating 360. In seme embodiments, the coating may coat the entire middle portion 340 of the stent expanding balloon 200. In some embodiments, the first taper region 325 may be coated. In some embodiments, the second taper region 335 may be coated. In some embodiments, the first portion 320 may be coated. In some embodiments, the second portion 330 may be coated. In some embodiments, the stent expanding balloon 300 may include a variety of different combination of the above noted coatings.

Similar to the stent expanding balloon 200, the stent expanding balloon 300 may comprise a friction enhancing features 310. In some embodiments, the fiction enhancing feature 310 may be similar to the micro or nano sized structures 250 and 260. In some embodiments, the friction enhancing feature 310 may be that the outer surface 301 is roughened as discussed above, or the outer surface 301 acquires a roughed surface from the interior of a mold.

In some embodiments, the friction enhancing feature 310 is dispersed over the outer surface 301. In another embodiment, the friction enhancing feature 310 is dispersed over the outer surface 301 of the middle portion 340, the first taper region 325, and the second taper region 335. In some embodiments, the friction enhancing feature 310 is dispersed only over the outer surface 301 of the middle portion 340. In some embodiments, the coating disclosed above is applied to the outer surface 301 of the stent expanding balloon on top of the friction enhancing feature 310. In other words, the first coating 350 is applied to the outer surface 301 with the friction enhancing feature 310 and the second coating 360 is applied to the outer surface 301 with the friction enhancing feature 310. Still further, in certain embodiments, a balloon may be configured with friction enhancing features such as those described above only in the portions of the balloon shown as having the first and second coatings 350, 360. In some instances, the friction enhancing features may be utilized instead of the coatings 350, 360 to provide extra friction in those regions.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of forming a medical balloon may include one or more of the following steps: extruding a first material to form a tube comprising a first material layer having an interior surface and an exterior surface; disposing a second material layer over the exterior surface of the medical balloon, wherein the second material layer is coupled to the exterior surface; and blow molding the tube in a mold to form a medical balloon. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially equivalent” is recited with respect to a feature, it is understood that in further embodiments, the feature can have a precisely equivalent configuration.

The terms “a” and “an” can be described as one, but not limited to one. For example, although the disclosure may recite a structure having “a seta,” the disclosure also contemplates that the housing can have two or more setae.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical balloon, comprising:
a balloon comprising:
a first tapered end portion;
a second tapered end portion; and
a body portion disposed between the first tapered end portion and the second tapered end portion, and comprising an exterior surface and an interior surface;
wherein the exterior surface comprises a friction enhancing feature configured to increase a static stent holding force.

2. The medical balloon of claim 1, wherein a first coefficient of friction of the exterior surface is greater than a second coefficient of friction of the interior surface.

3. The medical balloon of claim 1,
wherein the body portion further comprises a first material layer comprising the interior surface and the exterior surface,
wherein the friction enhancing feature comprises a second material layer disposed over the first material layer, and
wherein the second material layer is more compressible than the first material layer.

4. The medical balloon of claim 3, wherein the second material layer comprises one or more of silicone, polyurethane, polyether block amide, polytetrafluoroethylene, nylon, acrylic or methacrylic.

5. The medical balloon of claim 1, wherein an outer surface of the body portion is roughened.

6. The medical balloon of claim 1, wherein a first end of the body portion near the first tapered end portion comprises a first coating,
wherein a second end of the body portion near the second tapered end portion comprises a second coating, and
wherein an uncoated portion of the body portion is disposed between the first coating and the second coating.

7. The medical balloon of claim 6, wherein the first coating and the second coating have a durometer different than the uncoated portion.

8. The medical balloon of claim 6, wherein a durometer of the first coating and the second coating is greater than a durometer of the uncoated portion.

9. A stent expanding balloon, comprising:
a formed laminar balloon comprising:
a first material layer comprising an exterior surface and an interior surface; and
a friction enhancing feature configured to increase a static stent holding force,
wherein the friction enhancing feature comprises a second material layer disposed over the first material layer and coupled to the exterior surface.

10. The stent expanding balloon of claim 9, wherein the second material layer comprises a material having a Shore durometer of between 50 A and 80 D.

11. The stent expanding balloon of claim 9, wherein the second material layer comprises a material having a Shore durometer of about 56 D.

12. The stent expanding balloon of claim 9, wherein the second material layer comprises a first section near a first end of the balloon and a second section near a second end of the balloon with an uncoated section disposed between the first section and the second section.

13. The stent expanding balloon of claim 12, wherein a durometer of the first section and the second section of the second material layer is greater than a durometer of the uncoated section.

14. The stent expanding balloon of claim 13, wherein the durometer of the first section and the second section of the second material layer is between Shore durometer 50 A and 100 A.

15. The stent expanding balloon of claim 12, wherein the first section is configured to engage with a first end of a stent and the second section is configured to engage with a second end of the stent opposite the first end of the stent to reduce accordion of the stent during expansion of the stent.

16. A method of forming a medical balloon, comprising:
extruding a first material to form a tube comprising a first material layer having an interior surface and an exterior surface;
disposing a second material layer over the exterior surface, wherein the second material layer is coupled to the exterior surface; and
blow molding the tube in a mold to form a formed laminar balloon.

17. The method of claim 16, further comprising treating the exterior surface using one or more of dielectric barrier discharge, chemical priming, and mechanical texturing, prior to the step of disposing the second material layer over the exterior surface of the first material layer,
wherein the treated exterior surface has a water contact angle of less than 50 degrees.

18. The method of claim 16, wherein the exterior surface of the first material is roughened by sandblasting.

19. The method of claim 16, wherein an interior of the mold is roughened by sandblasting.

20. The method of claim 16, wherein the step of disposing the second material layer over the exterior surface of the first material comprises coating a first section of the first material near a first end of the medical balloon and coating a second section of the first material near a second end of the medical balloon and not coating a section between the first section and the second section.
